(19) United States
(12) Patent Application Publication
(10) Pub. No.: US 2025/0099458 A1
(43) Pub. Date: Mar. 27, 2025

(54) USE OF ANTI-IDIOPATHIC PULMONARY FIBROSIS DRUG NINTEDANIB IN TREATMENT OF TUBERCULOSIS

(71) Applicants: Beijing Chest Hospital, Capital Medical University, Tongzhou District, Beijing (CN); Beijing Tuberculosis Chest Cancer Institute, Tongzhou District, Beijing (CN)

(72) Inventors: Yu LU, ChaoYang District, Beijing (CN); Xiaoyou CHEN, ChaoYang District, Beijing (CN); Xueting QI, ChaoYang District, Beijing (CN); Luyao ZHENG, ChaoYang District, Beijing (CN); Lei FU, ChaoYang District, Beijing (CN); Weiyan ZHANG, ChaoYang District, Beijing (CN); Ning WANG, ChaoYang District, Beijing (CN)

(73) Assignees: Beijing Chest Hospital, Capital Medical University, Tongzhou District, Beijing (CN); Beijing Tuberculosis Chest Cancer Institute, Tongzhou District, Beijing (CN)

(21) Appl. No.: 18/727,731

(22) PCT Filed: Mar. 16, 2023
(86) PCT No.: PCT/CN2023/081772

§ 371 Date: Aug. 7, 2024

(30) Foreign Application Priority Data
Apr. 29, 2022 (CN) 202210463752.6

(51) Int. Cl. A61K 31/496; A61K 31/4409; A61K 31/4965; A61P 31/06

(52) U.S. Cl. CPC A61K 31/496; A61K 31/4409; A61K 31/4965; A61P 31/06

(57) ABSTRACT

Provided is a use of nintedanib or a pharmaceutically acceptable salt thereof in preparing a drug for treating tuberculosis. The nintedanib or the pharmaceutically acceptable salt thereof is used in combination with a further anti-tuberculosis drug for treating tuberculosis, or used as an adjuvant drug for the treating tuberculosis, where the further anti-tuberculosis drug is selected from a group consisting of: rifampicin, isoniazid, pyrazinamide, ethambutol, fluoroquinolone, streptomycin, kanamycin, amikacin, capreomycin, sodium para-aminosalicylate, ethionamide, cycloserine, clofazimine, and linezolid.

### TECHNICAL FIELD

The present disclosure relates to a novel use of a drug, and particularly relates to a use of nintedanib in preparation of a drug for treating tuberculosis.

### BACKGROUND

Tuberculosis (TB) is a chronic infectious disease caused by infection with Mycobacterium tuberculosis (MTB), and is still the single infectious disease that causes the greatest number of deaths so far. The Mycobacterium tuberculosis infection typically results in lesions characterized by granulomatous inflammation, destruction of the lung parenchyma, and interstitial fibrosis. The existing chemotherapy scheme has the problems of long treatment course, more side effects, poor efficacy and the like. Although standard antituberculosis treatment regimens are highly effective against drug-sensitive tuberculosis and can achieve microbiological cure, more than ⅔ of the treated patients have extensive lung structure changes, and more than a half have permanently impaired pulmonary functions. Tuberculosis-induced pulmonary fibrosis is a common pulmonary impairment after TB (PIAT), and extensive pulmonary fibrosis not only seriously affects pulmonary functions of the patient, but also causes tubercle bacillus wrapped by fibers, making it difficult to completely kill the bacteria by drugs and easy to relapse.

The Mycobacterium tuberculosis evolves with the human immune system, persists in infected cells in various ways, and causes serious pathology and tissue damage of the host, thereby reducing the therapeutic effect of the existing antibiotics and accelerating generation of drug resistance. In view of this, there is an urgent need for new therapeutic approaches and pharmaceutical composition regimens to deal with Mycobacterium tuberculosis infection. As an emerging treatment method in the anti-infective field, the host-directed therapy (HDT) is a new and effective adjuvant treatment method for tuberculosis. The HDT can (1) enhance the action of antibiotics, (2) shorten the duration of treatment for TB, (3) prevent relapse, and (4) improve the immunopathology, including TB-related matrix destruction and fibrosis which may interfere with the penetration and efficacy of antituberculosis drugs. Several studies have shown that cytokines such as tumor necrosis factor α (TNF-α), transforming growth factor β (TGF β) and interleukin 1β (IL-1β) mediate development of the fibrosis, and may lead to respiratory failure in patients with advanced chronic tuberculosis. This data underscores the need for an HDT method for collagen deposition and fibrosis formation.

Nintedanib (BIBF1120) is a potent intracellular tyrosine kinase inhibitor clinically used in the form of an ethyl sulfonate thereof with a structural formula of:

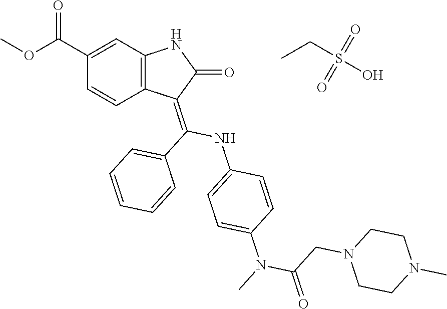

The current nintedanib ethyl sulfonate available in the market is a soft capsule that is clinically used for treatment of idiopathic pulmonary fibrosis (IPF). Current studies suggest that the transforming growth factor (TGF)-β, the platelet-derived growth factor (PDGF), the epidermal growth factor, the fibroblast growth factor (FGF), and the vascular endothelial growth factor (VEGF) all play important roles in the progression of fibrotic diseases, and can inhibit various signal transduction receptors related to the pathogenesis of fibrosis, including a platelet-derived growth factor receptor (PDGFR), a fibroblast growth factor receptor (FGFR), and a vascular endothelial growth factor receptor (VEGFR). Nintedanib inhibits PDGFR, FGFR and VEGFR, and thereby limits the effect of profibrotic mediators released from the damaged ECM. Nintedanib may have a dual beneficial effect of reducing both fibrosis and granuloma angiogenesis since it acts as a VEGFR inhibitor.

So far, no report about the application of nintedanib in tuberculosis treatment has been found at home and abroad yet.

It is found in related studies of the present disclosure that nintedanib can be used for treating tuberculosis.

### SUMMARY

In view of this, the present disclosure provides a use of nintedanib or a pharmaceutically acceptable salt thereof independently or in combination with a further anti-tuberculosis drug in preparation of a drug for treating tuberculosis.

In the use of the present disclosure, the pharmaceutically acceptable salt of nintedanib is a nintedanib ethanesulfonate salt.

The tuberculosis discussed in the present disclosure is an infectious disease caused by infection with Mycobacterium tuberculosis, which is called pulmonary tuberculosis when occurring in the lung, and called extrapulmonary tuberculosis when occurring outside the lung.

In the use of the present disclosure, the drug is used independently or in combination with a further anti-tuberculosis drug in treating tuberculosis, or used as an adjuvant drug for treating tuberculosis.

In the use of the present disclosure, the further anti-tuberculosis drug is selected from a group consisting of: rifampicin, isoniazid, pyrazinamide, ethambutol, fluoroquinolone, streptomycin, fluoroquinolone, kanamycin, amikacin, capreomycin, sodium para-aminosalicylate, ethionamide, cycloserine, clofazimine, and linezolid.

In the use of the present disclosure, the combined use includes a combined use of three or more types of drugs, or three or more types of drugs prepared into a compound pharmaceutical preparation.

In the use of the present disclosure, the dosage and administration of the nintedanib ethanesulfonate salt includes: 50 to 300 mg/d; orally administered 1 to 3 times daily.

In the use of the present disclosure, the drug is used at least 4 to 6 months for treating tuberculosis.

Another object of the present disclosure is to provide a pharmaceutical composition containing nintedanib and a further anti-tuberculosis drug, which is specifically prepared by mixing a nintedanib ethanesulfonate salt with isoniazid, rifampicin and pyrazinamide.

Based on analysis and evaluation of in vitro and mouse tuberculosis models, the present disclosure has accidentally found that the nintedanib of the present disclosure has an effect of treating tuberculosis. It is also found in further experiments that the combination of a nintedanib ethanesulfonate salt with rifampicin, or the combination of a nintedanib ethanesulfonate salt with isoniazid (H), rifampicin (R) and pyrazinamide, has an enhanced synergistic effect.

In view of this, the present disclosure preferably provides a combined use of a nintedanib ethanesulfonate salt with rifampicin, or a combined use of a nintedanib ethanesulfonate salt with isoniazid (H), rifampicin (R) and pyrazinamide, for treating tuberculosis. The combined use may include simultaneous or sequential administration of the drugs with their respective effective dose, for example, 150 mg for the nintedanib ethanesulfonate salt and 450 mg to 600 mg for rifampicin, 1 time daily. For the combined use of the nintedanib ethanesulfonate salt and isoniazid (H), rifampicin (R) and pyrazinamide (Z), their respective standard administration methods may be followed.

For easy administration, the present disclosure further provides a compound pharmaceutical preparation prepared by taking the nintedanib ethanesulfonate salt and rifampicin as active pharmaceutical ingredients. The compound pharmaceutical preparation may take the form of any oral preparation, such as tablets, capsules, granules or the like, or may be soft capsules. In the preparation of unit dose, effective doses of both drugs are contained. For example, each tablet contains 50 to 300 mg of the nintedanib ethanesulfonate salt, and 50 to 300 mg of rifampicin. Preferably, each tablet contains 150 mg of the nintedanib ethanesulfonate salt, and 450 mg to 600 mg of rifampicin.

The present disclosure further provides relevant experimental effects of nintedanib.

Compared with the existing pharmaceutical therapies for tuberculosis, the present disclosure has the advantages of:
\textcircled{1} novelty: no relevant report has found at home and abroad yet; and
\textcircled{2} an anti-tuberculosis activity is found in nintedanib for the first time;
\textcircled{3} shortened treatment course: for sensitive tuberculosis, the treatment course is expected to be shortened to 4 to 5 months;
\textcircled{4} good efficacy: the efficacy is equivalent to that of the existing solution; and
\textcircled{5} low recurrence: the recurrence rate is lower compared to the existing solution.

### BRIEF DESCRIPTION OF DRAWINGS

FIG. 1 shows CFU counts in lung tissues of individual treatment groups of lung tissues at different time points;

FIG. 2 shows hydroxyproline (HYP) contents of individual treatment groups at different time points;

FIG. 3 shows scores for 4 W treatment of alveolar inflammation, and scores for 8 W treatment of alveolar inflammation;

FIG. 4 shows Masson staining results: BIBF1120+HRZ treatment group vs. HRZ treatment group;

FIG. 5 shows ratios of Masson staining positive areas;

FIG. 6 shows a special staining positive ratio in each treatment group; and

FIG. 7 shows immunohistochemical results indicating that the CD31 expression amount is reduced in the BIBF1120+HRZ treatment group compared with the HRZ treatment group.

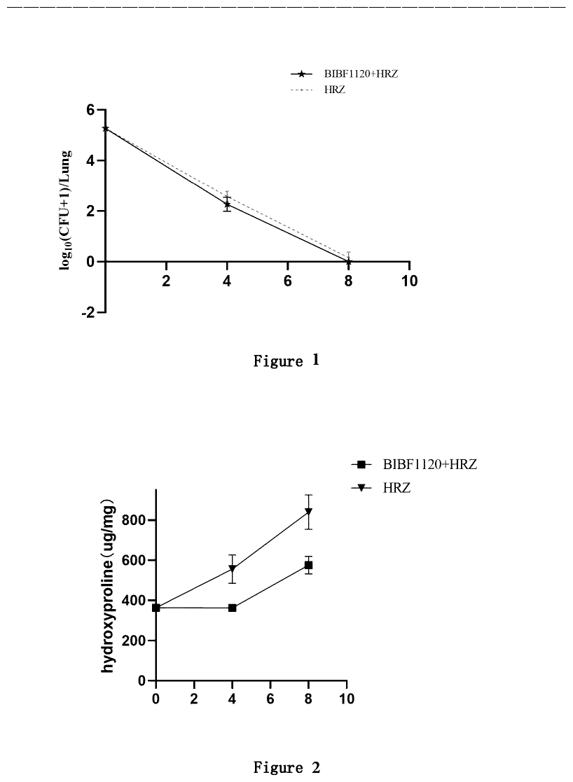

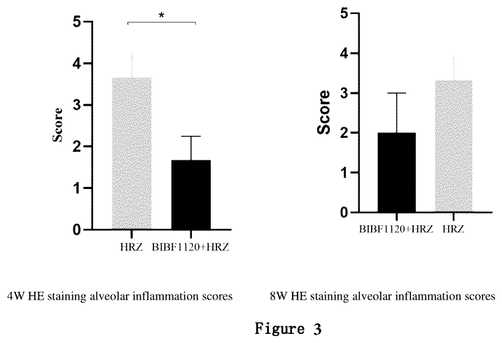

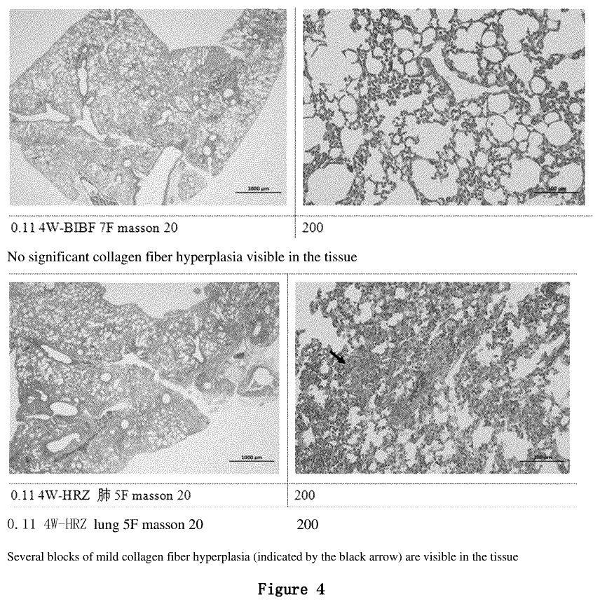

### DETAIL DESCRIPTION OF EMBODIMENTS

The present disclosure will be further described in detail below in conjunction with the drawings and embodiments, but the description is intended to explain, rather than limit, the present disclosure.

### Experimental Example 1: In Vitro Activity of Nintedanib on Mycobacterium tuberculosis

An H37Rv strain is cultured to the logarithmic growth phase, while a suspension is diluted to 1×10⁶ CFU/ml in a 7H9 liquid medium for later use. As shown in the following figure: columns A to F sequentially represent a positive/negative control well, INH, RFP, PFD, SC1011, and BIBF1120. Drug-containing wells are sequentially diluted in multiple proportions by a 96-well plate. After cultivation at 37° C. for 7 d, 20 μl of Alamar Blue and 12.5 μl of 20% Tween-80 are added to each well, further cultivation is performed at 37° C. for 24 h, and colors of the wells are recorded, where blue indicates no growth of the strain, red indicates growth of bacteria, and MIC indicates the lowest drug concentration for changing from blue to red. The experiment is repeated 3 times. The results show that: the nintedanib has an antibacterial activity, and the MIC value for an H37Rv standard strain is: 24.567 ug/ml.

### Experimental Example 2: Action of Nintedanib on Rifampicin as Anti-Tuberculosis Drug

A minimum drug concentration (MIC90) at which the growth of 90% Mycobacterium tuberculosis is inhabited is determined in a broth microdilution method, and then, based on the MIC value of each drug used alone, BIBF1120 and RFP are diluted to six concentrations, from 2×MIC to 1/16×MIC, in a two-fold dilution method. Wells at column 2 of the 96-well plate are each added with 50 μl of 2×MIC to 1/16×MIC A solution (BIBF1120), first 7 wells in rows 2 to 7 are each added with 50 μl of 2×MIC to 1/16×MIC B solution (RFP), each single-drug MIC assay well is added with 50 μl of 7H9, and finally, 100 μl of the diluted suspension is added to each well of the 96-well plate, while a positive control well (100 μl of diluted solution+100 μl of 7H9) and a negative control well (200 μl of 7H 9) are provided. The MICs of individual drugs in the combined use are determined by observing color changes in the 96-well plate (where a color changing from blue to purple or pink indicates bacterial growth; and blue indicates no bacterial growth due to inhibition of the drug). The combined function is determined by FICI, and FICI=(MICA combination/MICA alone)+ (MICB combination/MICB alone), where MICA alone and MICB alone respectively represent MICs of drug A and drug B when acting on Mycobacterium tuberculosis alone, and MICA combination and MICB combination respectively represent MICs of drug A and drug B when drug A and drug B used in combination reach the same efficacy as the drugs used alone. When FICI≤0.5, the two drugs have synergistic effects; when 0.5<FICI<1, the two drugs have partial synergistic effects; when FICI=1, the two drugs have additive effects; and when 1<FICI<4, the two drugs have unrelated effects, and when FICI>4, the two drugs have antagonistic effects. The results show that: the combination of the two drugs demonstrates antibacterial activity.

### Experimental Example 3: Macrophage Intracellular Bactericidal Activities of Different Concentrations of Nintedanib

The macrophage intracellular bactericidal activity is used to evaluate the anti-tuberculosis activities of BIBF1120 alone and BIBF1120+RFP in combination. The specific operating method includes: collecting J774A.1 macrophages into a 50 ml centrifuge tube, and taking 100 μl of macrophages and 900 μl of a cell culture medium, which are added into a 1.5 ml sterile centrifuge tube for 10-fold dilution; counting under a microscope; diluting macrophages to 4*10⁵/ml and gently plating the macrophages into a 48-well clear microwell plate, with 1 ml in each well; culturing in an incubator with 5% of CO₂ at 37° C. for 24 h after plating; co-culturing MOI=5:1 and 2*10⁶ CFU/MLH37Rv with the macrophages; rinsing 2 times with sterile PBS 4 h later to remove extracellular Mycobacterium tuberculosis, and adding a new RPMI cell culture medium; putting into an incubator with 5% of CO₂ at 37° C. and culturing for 72 h. discarding RPMI in the 48-well plate 3 days later, adding 200 μl of 0.1% SDS and placing in the incubator for 5 to 10 min, and adding 800 μl of the cell culture medium into each well and mixing well; and after cell lysis, taking 100 μl from each well for 10-, 100-, 1000-, 100000-fold dilution, and inoculating 100 μl on a 7H10 solid medium, spreading it evenly with a spreader and counting CFUs three weeks later.

Table 1 shows results of the intracellular bactericidal activity of BIBF1120 alone

| | CFU | log10 CFU |
|---|---|---|
| Blank control | 2400000 | 6.380 |
| RFP (2 ug/ml) | 502500 | 5.701 |
| BIBF1120 (50 ug/ml) | 1077500 | 6.032 |
| BIBF1120 (25 ug/ml) | 1462500 | 6.165 |
| BIBF1120 (12.5 ug/ml) | 2400000 | 6.380 |

Table 2 shows results of the macrophage intracellular bactericidal activity of BIBF1120+RFP in combination

| | CFU | log10 CFU |
|---|---|---|
| Blank control | 4600000 | 6.663 |
| RFP (2 ug/ml) | 202500 | 5.306 |
| BIBF1120 (25 ug/ml) | 2750000 | 6.439 |
| RFP (2 ug/ml) + BIBF1120 (25 ug/ml) | 48500 | 4.686 |
| RFP (2 ug/ml) + BIBF1120 (25 ug/ml) | 61500 | 4.789 |

The conclusion is that: macrophage intracellular experiments show that under the safe administration concentration, the BIBF1120 combined with the anti-tuberculosis drug RFP has an enhanced bactericidal effect. BIBF1120 has no significant bacteriostatic effect when administered alone at a dose of 25 ug/ml (similar to the in vitro MIC test result).

### Experimental Example 4: Effects of Nintedanib in Mouse Tuberculosis Model

An H37Rv strain in the logarithmic growth phase is diluted to a bacterial concentration of 1×10⁷ CFU/ml with 20 ml of 1×PBS, and 6 to 8 week-old C57BL/6 female mice are infected with aerosol. An aerosol infected chronic mouse tuberculosis model is created and used for evaluating the antibacterial activity of each treatment group. At day 10 after infection (D-32), 3 mice are sacrificed at random, and at the day of treatment administration (DO), 6 mice are sacrificed at random and dissected to take and count spleen and lung colony-forming units (CFU) on a 7H10 plate after spleen and lung tissue homogenate, so as to determine baseline pulmonary and splenic Mycobacterium tuberculosis in the mice at the beginning of infection and at the beginning of treatment. Administration is started 6 weeks after infection, and at weeks 4 and 8 after administration, 7 to 8 mice from each treatment group are randomly sacrificed for dissection, with spleen and lung colony-forming units (CFU) taken and counted on a 7H10 plate after spleen and lung tissue homogenate. After 8 weeks of treatment and 12 weeks off treatment, relapsing of pulmonary and splenic Mycobacterium tuberculosis in the 7 mice of each treatment group is observed, and C57BL/6 mice infected for 6 weeks are randomly divided into 3 groups (Table 3).

Table 3 shows CFU counts of lung tissues at different time points for each treatment group.

| | Group | | | |
|---|---|---|---|---|
| Time of | Control | HRZ + | HRZ + | |
| treatment | group | Pirfenidone | BIBF1120 | HRZ |
| D −32 | 4.38 ± 0.09 | | | |
| D 0 | 5.27 ± 0.066 | | | |
| 4 W | | 2.52 ± 0.23 | 2.27 ± 0.24 | 2.57 ± 0.18 |
| 8 W | | 0.30 (2/3) | 0 | 0.39 ± 0.09 (3/5) |
| W 8 + 12 W | | 6/7 | 2/7 | 5/7 |

Table 4 shows CFU counts of spleen at different time points for each treatment group.

| | Group | | | |
|---|---|---|---|---|
| Time of | Control | HRZ + | HRZ + | |
| treatment | group | Pirfenidone | BIBF1120 | HRZ |
| D −32 | 2.15 ± 0.11 | | | |
| D 0 | 3.496 ± 0.257 | | | |
| 4 W | | 1.82 ± 0.08 | 1.54 ± 0.10 | 1.79 ± 0.17 |
| 8 W | | 0 | 0 | 0 |
| W 8 + 12 W | | 5/7 | 1/7 | 5/7 |

Annotation: isoniazid (H); rifampicin (R); pyrazinamide (Z); D-32: day 10 after infection; W4: 4 weeks of treatment; W8: 8 weeks of treatment; W8+12 W: observing relapsing after 8 weeks of treatment and 12 weeks off treatment. D: day; W: week; isoniazid: 10 mg/kg/d; rifampicin: 10 mg/kg/d; pyrazinamide: 150 mg/kg/d; Pirfenidone (PFD): 100 mg/kg/d; BIBF1120: 50 mg/kg/d; where RFP is administered at a minimum interval of 1 h with other drugs

### Conclusion

1. The BIBF1120+HRZ treatment group and the HRZ treatment group both have significant bactericidal effects after administration for 4 weeks and 8 weeks, where the BIBF1120 achieves sterilizing of the spleen and the lung after 8 weeks of administration, and further, the addition of BIBF1120 can shorten the treatment time of pulmonary tuberculosis and achieve sterilizing earlier.
2. The BIBF1120+HRZ treated group has a reduced recurrence rate in mice compared with the HRZ treated group.

### Experimental Example 5

assay with HYP assay kit. A hydroxyproline content in the right lung tissue is assayed. A fresh lung tissue is dissected and separated, weighed, and placed into a test tube, and added with precisely 1 ml of hydrolysate. The tissue is placed in a water bath kettle for 20 min of water bath. The pH is adjusted to about 6.0 to 6.8. Then, distilled water is added to 10 ml, and 4 ml of diluted hydrolysate is taken and added with a proper amount of activated carbon. Centrifuging is performed at 3500 r/min for 10 min, and 1 ml of supernatant is taken for detection. A blank tube and a standard tube represent distilled water and a standard, respectively. Reagents are added sequentially according to instructions. Finally, an absorbance value A of each tube is assayed by a microplate reader with distilled water set to zero at 550 nm. The HYP content is calculated by: HYP content (μg/mg wet weight)=(A_{assay}−A_{blank})/(A_{standard}−A_{blank})*5 μg/ml (standard tube content)*[10 (total hydrolysate volume)/wet weight of lung tissue]. A_{assay}: the assayed absorbance of tube; A_{blank}: the absorbance of the blank tube; and A_{standard}: the absorbance of the standard tube. the absorbance of the blank tube; and A_{standard}: the absorbance of the standard tube.

Table 5 shows Hyp contents in the lung tissue at different treatment time points.

### Analysis of HE Staining Results

| | | | HRZ + |
|---|---|---|---|
| | Control | HRZ | BIBF1120 |
| D 0 | 362.99 ug/mg | | |
| D4W | | 636.141 | 363.891 |
| | | 524.829 | 345.454 |
| | | 505.488 | 378.183 |
| Mean | | 555.486 ± 57.58 | 362.509 ± 13.39 |
| D8W | | 907.965 | 588.994 |
| | | 743.676 | 526.047 |
| | | 866.896 | 611.289 |
| Mean | | 839.512 ± 69.810 | 575.443 ± 36.095 |

### Pathological Observations of the Lung Tissue

A left lung tissue of a mouse is taken for trimming, and fixed by 4% paraformaldehyde. After being fixed well, the tissue is rinsed with physiological saline, fixed in a 4% paraformaldehyde solution, embedded by a conventional paraffin, and prepared into paraffin slices of 3 μm. Then, HE staining is performed to observe pathological changes in the lung tissue, and the pathological changes in the lung tissue is observed under a light microscope to describe an inflammatory infiltration degree and perform pathological scoring for lung injury. The degree of lung injury is assessed by 4 indices: 1 alveolar congestion; 2 alveolar hemorrhage; 3 neutrophil infiltration or aggregation in alveolar spaces or vascular walls; and 4) thickened alveolar septum. The scoring criteria include: 0 score for no injury, 1 score for mild injury, 2 scores for moderate injury, and 3 scores for severe injury, where all the scores are summed to obtain a pathological score for lung injury.

Table 6 shows alveolar inflammation scores after 4 W treatment.

| | Pathological change | | | | |
|---|---|---|---|---|---|
| | | | Neutrophil | | |
| | | | infiltration or | | |
| | | | aggregation | | |
| | | | in alveolar | | |
| | | | spaces or | Thickened | Pathological |
| | Alveolar | Alveolar | vascular | alveolar | score for |
| No. | congestion | hemorrhage | walls | septum | lung injury |
| 10.114W-BIBF 5F | 0 | 0 | 1 | 0 | 1 |
| 10.114W-BIBF 6F | 0 | 2 | 0 | 0 | 2 |
| 10.114W-BIBF 7F | 1 | 1 | 0 | 0 | 2 |
| 10.114W-HRZ lung 5F | 0 | 1 | 1 | 2 | 4 |
| 10.114W-HRZ lung 6F | 0 | 1 | 1 | 2 | 3 |
| 10.114W-HRZ lung 7F | 0 | 1 | 1 | 1 | 3 |

Table 7 shows alveolar inflammation scores after 8 W treatment.

| | Pathological change | | | | |
|---|---|---|---|---|---|
| | | | Neutrophil | | |
| | | | infiltration or | | |
| | | | aggregation | | |
| | | | in alveolar | | |
| | | | spaces or | Thickened | Pathological |
| | Alveolar | Alveolar | vascular | alveolar | score for |
| No. | congestion | hemorrhage | walls | septum | lung injury |
| B-1 lung | 0 | 1 | 1 | 0 | 2 |
| B-2 lung | 0 | 1 | 1 | 1 | 3 |
| B-3 lung | 0 | 1 | 0 | 0 | 1 |
| H-1 lung | 0 | 1 | 1 | 2 | 4 |
| H-2 lung | 0 | 0 | 1 | 2 | 3 |
| H-3 lung | 0 | 1 | 1 | 2 | 4 |

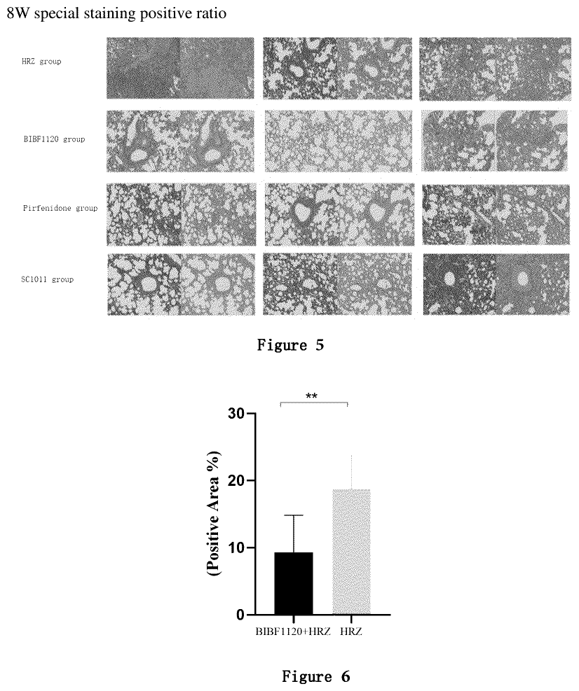

Masson staining is performed in a method according to instructions of the kit. Slices are dewaxed conventionally to water, soaked overnight in Masson A solution, soaked for 1 min in a combination of Masson B solution and Masson C solution mixed in equal proportions, soaked for 6 min in Masson D solution, soaked for 1 min in Masson E solution, soaked for 2 to 30 s in Masson F solution, rinsed and differentiated with 1% glacial acetic acid, dehydrated with absolute ethanol, clarified in xylene, sealed with a neutral gum, and observed under a normal optical microscope. The results show that collagen fibers appear blue; and myofibers, cellulose and red blood cells appear red. An imaging system is used to collect images on the stained tissue slices, while analysis software is used to read a measured tissue region automatically, calculate a positive area and a tissue area in the measured region, and thereby calculate a proportion of the positive area, as shown in FIGS. 5 and 6.

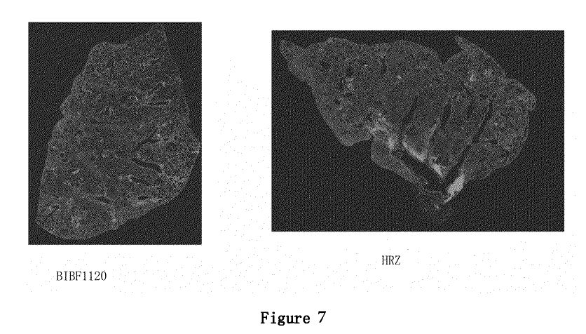

After being rewarmed, frozen slices are rinsed 3 times with a PBS buffer and then added with sodium citrate. When the slices are naturally cooled and rinsed, 5% of goat serum for sealing is added to seal the slices for 2 hours at room temperature. After the sealing is completed, the slices are added with an anti-CD31 antibody and incubated overnight at 4° C., where the antibody concentration is 1:200. At day 2, the slices are added with a biotin-labeled secondary antibody, incubated for 1 h at room temperature, and rinsed for 3 times, and then added with a horseradish peroxidase-labeled streptavidin working solution, incubated for 15 min at 37° C., developed with DAB, counterstained with hematoxylin, sealed, and imaged and recorded with a biological microscope, as shown in FIG. 7, where immunohistochemical results indicate that the CD31 expression amount is reduced in the BIBF1120+HRZ treatment group compared with the HRZ treatment group. It is preliminarily speculated that the adjuvant anti-tuberculosis therapeutic effect of BIBF1120 may be enabling a better anti-tuberculosis activity by inhibiting abnormal vascular proliferation and improving delivery of small molecular compounds (anti-tuberculosis drugs).

1. A use of nintedanib or a pharmaceutically acceptable salt thereof independently or in combination with a further anti-tuberculosis drug in preparation of a drug for treating tuberculosis.

2. The use according to claim 1, wherein the pharmaceutically acceptable salt of nintedanib is a nintedanib ethanesulfonate salt.

3. The use according to claim 1, wherein the tuberculosis is pulmonary tuberculosis or extrapulmonary tuberculosis.

4. The use according to claim 1, wherein the tuberculosis is pulmonary tuberculosis.

5. The use according to claim 1, wherein the nintedanib or the pharmaceutically acceptable salt thereof is used independently or in combination with a further anti-tuberculosis drug in treating tuberculosis, or used as an adjuvant drug for treating tuberculosis.

6. The use according to claim 5, wherein the further anti-tuberculosis drug is selected from a group consisting of: rifampicin, isoniazid, pyrazinamide, ethambutol, fluoroquinolone, streptomycin, kanamycin, amikacin, capreomycin, sodium para-aminosalicylate, ethionamide, cycloserine, clofazimine, and linezolid.

7. The use according to claim 6, wherein the combined use includes a combined use of three or more types of drugs, or three or more types of drugs prepared into a compound pharmaceutical preparation.

8. The use according to claim 7, wherein the compound pharmaceutical preparation is prepared by mixing a nintedanib ethanesulfonate salt with isoniazid, rifampicin and pyrazinamide.

9. The use according to claim 1, wherein the dosage and administration of the nintedanib ethanesulfonate salt includes: 50 to 300 mg/d; orally administered 1 to 3 times daily, at least 4 to 6 months for treating tuberculosis.

10. A pharmaceutical composition containing nintedanib and a further anti-tuberculosis drug, characterized in that the pharmaceutical composition is prepared by mixing a nintedanib ethanesulfonate salt with isoniazid, rifampicin and pyrazinamide.
